(19) United States
(12) Patent Application Publication
(10) Pub. No.: US 2002/0088044 A1
(43) Pub. Date: Jul. 11, 2002

(54) Self-sizing sun visor

(76) Inventor: Razgo Lee, Diamond Bar, CA (US)

Correspondence Address: CHAN LAW GROUP LC, 1055 W. 7TH ST,, SUITE 1880, LOS ANGELES, CA, 90017, US

(21) Appl. No.: 09/881,140

(22) Filed: Jun. 14, 2001

Related U.S. Application Data
(60) Provisional application No. 60/261,105, filed on Jan. 11, 2001.

(51) Int. Cl. A61F 9/00

(52) U.S. Cl. 2/12; 2/183

(57) ABSTRACT

The self-sizing sun visor fills the need for a visor that automatically adjusts to the wearer's head without causing undo compressive pressure on the wearer's head. The desired level of comfort and easy of use to the wearer is achieved by constructing the headband at least partially from stretchable material and incorporating a sweat absorbing foam inner layer. A bill of essentially a rigid nature is attached to the forward portion of the lower peripheral edge. The headband can also be constructed from two or more headband segments. A headband segment is a different color or the same color as the other headband segments. The visor comes in a small/medium size and a large/extra large size to accommodate a range of head sizes comfortably.

### BACKGROUND OF THE INVENTION

1. Field of the Invention

The present invention relates to an improved construction of a self-sizing sun visor, capable of fitting wearers having a plurality of head sizes. The visor includes an open crown portion including a biaxially stretchable headband, a visor portion connected to the front of the headband and a sweat absorbing foam liner attached to the inner surface of the headband. The biaxially stretchable headband and the sweat absorbing foam liner attached to the inner surface of the headband allows the visor to fit onto the head of the wearer without uncomfortable circumferential pressure.

2. Description of Related Art

Visors are well known in the art and come in many configurations. They include a headband with an open crown to fit onto a wearer's head and a visor or bill portion attached to the front of the headband.

There are many different types of visors that have been available over the years. In order to fit a plurality of head sizes prior art visor headbands took the form of a rigid headband with an open back end, a stretchable continuous loop headband or a headband with wearer adjustable overlapping straps.

The desired level of comfort and easy of use to the wearer has not been achieved in the prior art visors which incorporate some type of stretchable fabric headband. The visor causes undo compressive pressure around the head of the wearer thereby causing discomfort to the wearer and even resulting in a mark being left on the wearer's skin.

An objective of the present invention is to overcome the disadvantages of the prior art by providing a custom-fit, comfortable visor having no adjustable straps at the back thereof and yet is capable of accommodating a plurality of various sized heads. It is a further objective of the present invention that it can be manufactured easily, utilizing conventional materials and fabrics.

Yet another objective is a visor that is both functional and attractive in its use and appearance. necessary for a retailer to maintain in order to accommodate a plurality of various sized heads.

### BRIEF SUMMARY OF THE INVENTION

The invention is a self-sizing sun visor. The self-sizing sun visor includes a headband. The headband has one or more layers of material. The headband is shaped as an elongated fouredge band and has an upper edge, a lower edge, a first edge, a second edge, a forward headband midpoint, an inner surface, an outer surface and a first predetermined width. The headband is constructed from one or more elongated rectangular pieces. The first edge of the headband is attached to the second edge of the headband, thus forming a headband with an inner surface, an outer surface and a circumferential linear extent of a first predetermined length. The headband has an upper peripheral edge and a lower peripheral edge.

A visor of an essentially rigid nature is attached to the forward portion of the lower peripheral edge.

In a variation of the invention the headband is at least uniaxially stretchable.

In another variant of the invention, the headband is at least biaxially stretchable.

In yet another variation of the invention, a sweat absorbing foam liner is attached to the inner surface of the headband to facilitate a comfortable snug fit.

In still another variation of the invention, the first predetermined length of the headband is a short length sized to fit small to medium size heads comfortably.

In a distinct variation on the invention, the first predetermined length of the headband is a long length sized to fit large to extra large size heads comfortably.

In another distinct variation on the invention, the headband is made from a stretchable cotton and spandex blend.

In another distinct variation on the invention, the first predetermined width of the headband decreases from a maximum width at the forward headband midpoint to a minimum width at the aft midpoint of the headband.

In still another distinct variation on the invention, the predetermined width of the headband increases from a maximum width at the aft headband midpoint to a minimum width at the forward midpoint of the headband.

In another distinct version of the invention, at least one edge of said elongated four-edge band is curvilinear.

In still another distinct version of the invention, at least one edge of said elongated fouredge band is straight.

In yet another distinct variation on the invention, the headband is created from one or more headband segments. The headband segment is shaped as an elongated four-edge band with an upper edge, a lower edge, a first edge, a second edge and a first predetermined width. The first edge of the headband segment is attached to the second edge of an adjacent headband segment and so forth to create a closed loop one or more piece, one or more layer headband. The headband has an inner surface, an outer surface and a circumferential linear extent of a first predetermined length.

In another version of the invention, the sweat absorbing foam liner is attached to the inner surface of the headband with stretchable thread.

In a different version of the invention, a headband segment is a different color or the same color as the other headband segments.

In yet a different version of the invention, a headband segment is sweat absorbent or nonsweat absorbent.

In still a different version of the invention, the headband is capable of construction from a mixture of more than two headband segments. The mixture including a uniaxially stretchable segment, a biaxially stretchable segment and or non-stretchable segment.

In another distinctive version of the invention, at least one edge of said elongated fouredge band is curvilinear.

In still another distinctive version of the invention, at least one edge of said elongated four-edge band is straight.

### BRIEF DESCRIPTION OF THE DRAWINGS

FIG. 1 is an orthogonal view from the bottom looking upwards into the inside of the visor.

### DETAILED DESCRIPTION

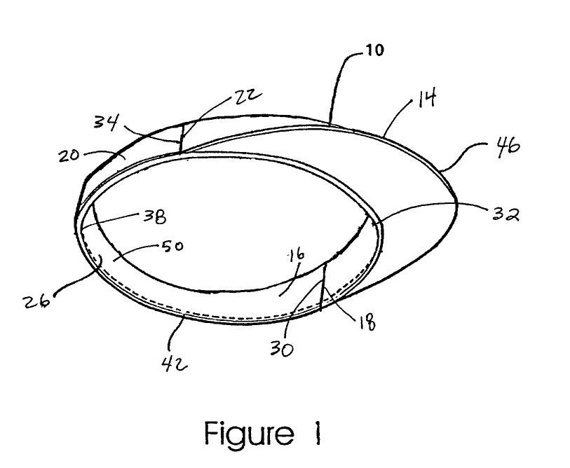

As shown in FIG. 1, the invention is a self-sizing sun visor 10. The self-sizing sun visor 10 includes a headband. The headband 14 has one or more layers 16 and 20 of material. The headband 14 is shaped as an elongated four edge band and has an upper edge, a lower edge, a first edge 18, a second edge 22, a forward headband midpoint 32, an inner surface 26, an outer surface and a first predetermined width. The headband is constructed from one or more elongated rectangular pieces. The first edge of the headband is attached to the second edge of the headband, thus forming a headband with an inner surface, an outer surface and a circumferential linear extent of a first predetermined length. The headband has an upper peripheral edge and a lower peripheral edge 42.

A visor 46 of an essentially rigid nature is attached to the forward portion of the lower peripheral edge 42.

In a variation of the invention the headband 14 is at least uniaxially stretchable.

In another variant of the invention, the headband 14 is at least biaxially stretchable.

In yet another variation of the invention, a sweat absorbing foam liner 50 is attached to the inner surface 26 of the headband to facilitate a comfortable snug fit.

In still another variation of the invention, the first predetermined length of the headband 14 is a short length sized to fit small to medium size heads comfortably.

In a distinct variation on the invention, the first predetermined length of the headband 14 is a long length sized to fit large to extra large size heads comfortably.

In another distinct variation on the invention, the headband 14 is made from a stretchable cotton and spandex blend.

In another distinct variation on the invention, the first predetermined width of the headband 14 decreases from a maximum width at the forward headband midpoint 32 to a minimum width at the aft midpoint 38 of the headband.

In still another distinct variation on the invention, the predetermined width of the headband increases from a maximum width at the aft headband midpoint 38 to a minimum width at the forward headband midpoint 32.

In another distinct version of the invention, at least one edge of said elongated four-edge band is curvilinear.

In still another distinct version of the invention, at least one edge of said elongated fouredge band is straight.

In yet another distinct variation on the invention, the headband 14 is created from one or more headband segments. The headband segment is shaped as an elongated four-edge band with an upper edge, a lower edge, a first edge 18 and 34, a second edge 22 and 30 and a first predetermined width. The first edge 18 of the headband segment is attached to the second edge 30 of an adjacent headband segment and so forth to create a closed loop one or more piece, one or more layer headband 14. The headband 14 has an inner surface 26, an outer surface and a circumferential linear extent of a first predetermined length.

In another version of the invention, the sweat absorbing foam liner 50 is attached to the inner surface of the headband with stretchable thread.

In a different version of the invention, a headband segment is a different color or the same color as the other headband segments.

In yet a different version of the invention, a headband segment is sweat absorbent or nonsweat absorbent.

In still a different version of the invention, the headband is capable of construction from a mixture of more than two headband segments. The mixture including a uniaxially stretchable segment, a biaxially stretchable segment and or non-stretchable segment.

In another distinctive version of the invention, at least one edge of said elongated fouredge band is curvilinear.

In still another distinctive version of the invention, at least one edge of said elongated four-edge band is straight.

What is claimed is:

1. A self-sizing sun visor, comprising:
a headband being provided, said headband having one or more layers of material, said headband being shaped as an elongated four edge band and having an upper edge, a lower edge, a first edge, a second edge, a forward headband midpoint, an inner surface, an outer surface and a first predetermined width, said headband being constructed from one or more elongated rectangular pieces;
said first edge of said headband being attached to said second edge of said headband, thus forming a headband loop with an inner surface, an outer surface and a circumferential linear extent of a first predetermined length, said headband having an upper peripheral edge and a lower peripheral edge; and
a visor of an essentially rigid nature being attached to said forward portion of said lower peripheral edge.

2. A self-sizing sun visor according to claim 1 wherein said headband being at least uniaxially stretchable.

3. A self-sizing sun visor according to claim 1 wherein said headband being at least biaxially stretchable.

4. A self-sizing sun visor according to claim 1 wherein a sweat absorbing foam liner being attached to said inner surface of said headband to facilitate a comfortable snug fit.

5. A self-sizing sun visor according to claim 1 wherein said first predetermined length of said headband being a short length sized to fit small to medium size heads comfortably.

6. A self-sizing sun visor according to claim 1 wherein said first predetermined length of said headband being a long length sized to fit large to extra large size heads comfortably.

7. A self-sizing sun visor according to claim 1 wherein the material said headband being made from being a stretchable cotton and spandex blend.

8. A self-sizing sun visor according to claim 1 wherein said first predetermined width of said headband decreases from a maximum width at said forward headband midpoint to a minimum width at the aft midpoint of said headband.

9. A self-sizing sun visor according to claim 1 wherein said first predetermined width of said headband increases from a maximum width at the aft headband midpoint to a minimum width at said forward midpoint of said headband.

10. A self-sizing sun visor according to claim 1 wherein at least one edge of said elongated fouredge band being curvilinear.

11. A self-sizing sun visor according to claim 1 wherein at least one edge of said elongated fouredge band being straight.

12. A self-sizing sun visor according to claim 1 wherein said headband being created from one or more headband segments, said headband segment being shaped as an elongated four edge band with an upper edge, a lower edge, a first edge, and a second edge and a first predetermined width;
said first edge of said headband segment being attached to said second edge of an adjacent said headband segment and so forth to create a closed loop a one or more piece, one or more layer said headband, said headband having an inner surface, an outer surface and a circumferential linear extent of a first predetermined length.

13. A self-sizing sun visor according to claim 4 wherein said sweat absorbing foam liner being attached to said inner surface of said headband with stretchable thread.

14. A self-sizing sun visor according to claim 12 wherein a said headband segment being a different color from the other said headband segments.

15. A self-sizing sun visor according to claim 12 wherein a said headband segment being the same color as the other said headband segments.

16. A self-sizing sun visor according to claim 12 wherein a said headband segment being sweat absorbent or non-sweat absorbent.

17. A self-sizing sun visor according to claim 12 wherein said headband being capable of construction of a mixture of more than two said headband segments, said mixture including a uniaxially stretchable segment, a biaxially stretchable segment and or nonstretchable segment.

18. A self-sizing sun visor according to claim 12 wherein at least one edge of said elongated four-edge band being curvilinear.

19. A self-sizing sun visor according to claim 12 wherein at least one edge of said elongated four-edge band being straight.
